(19) United States
(12) Patent Application Publication
(10) Pub. No.: US 2022/0378283 A1
(43) Pub. Date: Dec. 1, 2022

(54) Lighted Mirror System Utilizing Fluorescence Enhanced Theragnosis

(71) Applicant: Designs for Vision, Inc., Bohemia, NY (US)

(72) Inventors: Richard E. Feinbloom, New York, NY (US); Gal Hitch, Rishon Leziyyon (IL); Liviu Steier, Needham, MA (US)

(21) Appl. No.: 17/825,672

(22) Filed: May 26, 2022

Related U.S. Application Data
(60) Provisional application No. 63/272,115, filed on Oct. 26, 2021.
(60) Provisional application No. 63/217,953, filed on Jul. 2, 2021.
(60) Provisional application No. 63/193,734, filed on May 27, 2021.

(51) Int. Cl. A61B 1/06; A61B 1/247

(52) U.S. Cl. CPC A61B 1/0638; A61B 1/247

(57) ABSTRACT

A hand-held mirror assembly using Fluorescence Enhanced Theragnosis suitable for use in dental arts is shown wherein the assembly including a body containing at least one lighting source configured to emit a light that is directed toward an object and at least one reflective surface configured to receive a reflection of the light from the object and a filter system configured to block from the reflected light the emitted light while allowing light caused by the interaction of the emitted light with the object to be viewable.

### CLAIM OF PRIORITY

This application claims, pursuant to 35 USC 119, priority to, and the benefit of the earlier date of, patent application Ser. Nos. 63/272,115 filed on Oct. 26, 2021, 63/217,953 filed on Jul. 2, 2021 and 63/193,734 filed on May 27, 2021, the contents of all of which are incorporated by reference, herein

### BACKGROUND OF THE INVENTION

### Field of the Invention

The invention is related to the field of optics and in particular to a Lighted Mirror System for applying fluorescent enhanced theragnosis (FET) technology in the examination of a body cavity.

### Background Information

Fluorescent enhance theragnosis (FET) technology has emerged as a new tool that allows for the identification of healthy tissue from diseased tissue. Particularly, FET utilizes light technology in known wavelength bands to illuminate bacteria or infection in tissue, wherein the bacteria may generate a light in another wavelength band (i.e., a fluorescent light) that may be viewed by the practitioner. The location of the fluorescent light provides the practitioner the ability to provide a more complete care in resolving issues that may not otherwise be seen by the practitioner.

Mirror type systems are well-known tools utilized by dental practitioners for the inspection of areas of a patient's teeth that are not directly viewable by the practitioner. Generally, a small circular mirror, attached to a handle, may be inserted into a patient's mouth so that the practitioner may view the conditions in and around a patient's teeth. The practitioner may further use the mirror system to monitor the work being performed on the patient.

Similarly, medical practitioners may utilize mirror-type systems to view body cavities of patients when the area to be viewed is not directly accessible to the practitioner. For instance, a practitioner may utilize a mirror system to view a portion of an ear canal that is not directly in the view of the doctor. Mirror systems may be used in other more fields of medicine to observe portions of body cavities or other types of openings in the body, such as wounds, that are not normally directly viewable by the practitioner.

Hence, there is a significant advantage for the development of a mirror type system using FET in the examination and treatment in dental and surgical arts.

However, the wavelength band(s) of the light used in FET are in wavelength ranges or bands that may be hazardous to the eyes of a practitioner if the practitioner were to view the FET light directly. In the case of dental/medical procedures, where the practitioner utilizes a mirror to view areas of the patient's body cavity (e.g., mouth in the case of a dentist) or opening (e.g., punctures or wounds), the mirror may reflect light that may cause harm to the practitioner's eyes.

Hence, there is a need in the industry for a system that enables a practitioner to utilize FET technology during an examination of a patient and possibly the identification of a pathogenic microbiome while concurrently providing protection to the practitioner.

### SUMMARY OF THE INVENTION

It is an object of the instant invention to provide a system that utilizes a fluorescent enhanced theragnosis (FET) technology in the examination of a body cavity of a patient.

A system for utilizing fluorescent enhanced theragnosis (FET) technology in dental and/or medical procedures is disclosed, wherein the system comprises at least one mirror assembly configured to reflect images of a patient's cavity and at least one assembly configured to generate at least one light into the patient's cavity, wherein the at least one light causes a fluorescent light to be generated within the cavity. The generated fluorescent light may indicate the presence of bacteria or infection that may not be otherwise visible.

A lighted mirror system utilizing fluorescent enhanced theragnosis (FET) technology is disclosed, wherein the lighted mirror system includes a first lighting source configured to light a body cavity of a patient and a second lighting source configured to cause a fluorescent light to be generated within the body cavity, wherein the second light causing the generation of the fluorescent light is prevented from being viewed by a user.

A lighted mirror system is disclosed utilizing fluorescent enhanced theragnosis (FET) technology, wherein a lighting source is configurated to be projected into a body cavity of a patient that causes the generation of a fluorescent light by bacteria or infection within the body cavity.

A lighted mirror system is disclosed utilizing fluorescent enhanced theragnosis (FET) technology, wherein at least one lighting source projects light within a body cavity, which causes the generation of a fluorescent light and an image capture system that captures the fluorescence light for viewing and recording.

A lighting system that may be fitted to a mirror system is disclosed wherein the lighting system emits a light that may undercover the presence of bacterial and/or infection through the generation of a fluorescent light.

For a better understanding of exemplary embodiments and to show how the same may be carried into effect, reference is made to the accompanying drawings. It is stressed that the particulars shown are by way of example only and for purposes of illustrative discussion of the preferred embodiments of the present disclosure and are presented to clarify the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

### BRIEF DESCRIPTION OF THE DRAWINGS

The advantages, nature, and various additional features of the invention will appear more fully upon consideration of the illustrative embodiments described in detail in connection with the accompanying drawings, where like or similar reference numerals are used to identify like or similar elements throughout the drawings:

FIG. 1 illustrates of a side view of a first exemplary embodiment of a Lighted Mirror System in accordance with the principles of the invention.

FIG. 2 illustrates a perspective view of the Lighted Mirror System shown in FIG. 1 in accordance with the principles of the invention.

FIG. 3 illustrates a second perspective view of the Lighted Mirror System shown in FIG. 1 in accordance with the principles of the invention.

FIG. 4 illustrates a side view of a second exemplary embodiment a Lighted Mirror System in accordance with the principles of the invention.

FIG. 5 illustrates a perspective view of the Lighted Mirror System shown in FIG. 4 in accordance with the principles of the invention.

FIG. 6 illustrates a top view of a third exemplary embodiment of a Lighted Mirror System in accordance with the principles of the invention.

FIG. 7 illustrates a side view, through section B-B, of the third exemplary embodiment of a Lighted Mirror System shown in FIG. 8A, in accordance with the principles of the invention.

FIG. 8A illustrates an exploded side view of the third exemplary embodiment of a Lighted Mirror System shown in FIGS. 6 and 7 in accordance with the principles of the invention.

FIG. 8B illustrates an expanded view of the area identified as FIG. 8B in the third exemplary embodiment of the Lighted Mirror System shown in FIG. 8A.

FIG. 9A illustrates a top view of a fourth exemplary embodiment of a Lighted Mirror System in accordance with the principles of the invention.

FIG. 9B illustrates a side view, through section C-C of the fourth exemplary embodiment of the Lighted Mirror System shown in FIG. 9A in accordance with the principles of the invention.

FIG. 9C illustrates an expanded view within the area identified as FIG. 9C in the fourth exemplary embodiment of the Lighted Mirror System shown in FIG. 9B.

FIG. 9D illustrates a perspective view of the fourth exemplary embodiment of the Lighted Mirror System shown in FIG. 9A.

FIG. 10 illustrates a second side view of the fourth exemplary embodiment of the Lighted Mirror System shown in FIG. 9A.

FIG. 11A illustrates a side view of a second aspect of the fourth exemplary embodiment of the Lighted Mirror System shown in FIG. 9D.

FIG. 11B illustrates an expanded view of the area designated as FIG. 11B in FIG. 11A.

FIG. 12A illustrates a side view of a third aspect of the fourth exemplary embodiment of the Lighted Mirror System shown in FIG. 9A.

FIG. 12B illustrates an expanded view of the area designated as FIG. 12B in FIG. 12A.

FIG. 13A illustrates a side view of a fourth aspect of the fourth exemplary embodiment of the Lighted Mirror System shown in FIG. 9A.

FIG. 13B illustrates an expanded view of the area designated as FIG. 13B in FIG. 13A.

FIG. 14A illustrates a side view of a fifth aspect of the fourth exemplary embodiment of the Lighted Mirror System shown in FIG. 9A.

FIG. 14B illustrates an expanded view of the area designated as FIG. 14B in FIG. 14A.

FIG. 15A illustrates a side view of a sixth aspect of the fourth exemplary embodiment of the Lighted Mirror System shown in FIG. 9A.

FIG. 15B illustrates an expanded view of the area designated as FIG. 15B in FIG. 15A.

FIG. 16 illustrates a perspective view of an exemplary embodiment of a clip-on filter in accordance with the principles of the invention.

FIG. 17 illustrates of an exploded perspective view of a fifth exemplary embodiment of a Lighted Mirror system in accordance with a the principles of the invention.

FIG. 18 illustrates a cross-sectional view of the fifth exemplary embodiment of the Lighted Mirror system shown in FIG. 17 in accordance with the principles of the invention.

It is to be understood that the figures, which are not drawn to scale, and descriptions of the present invention described herein have been simplified to illustrate the elements that are relevant for a clear understanding of the present invention, while eliminating, for purposes of clarity, many other elements. However, because these omitted elements are well-known in the art, and because they do not facilitate a better understanding of the present invention, a discussion of such elements are not provided herein. The disclosure, herein, is directed also to variations and modifications known to those skilled in the art.

### BRIEF DESCRIPTION OF THE INVENTION

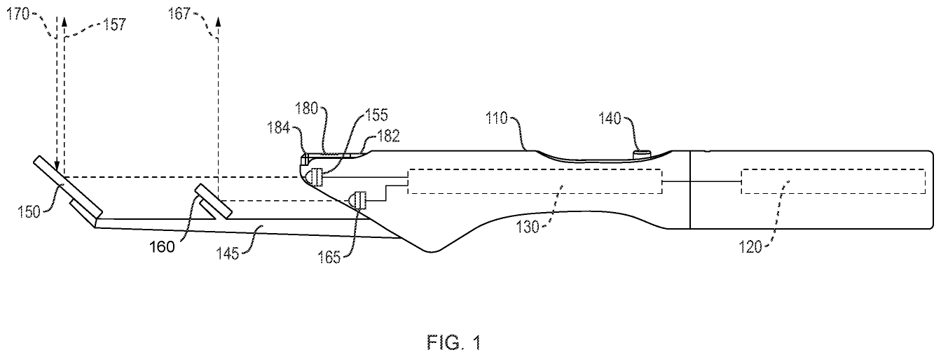

FIG. 1 illustrates a side view of a first exemplary embodiment of a Lighted Mirror System 100 in accordance with the principles of the invention.

In this illustrated embodiment, lighted mirror system 100 comprises a body or housing 110 and an arm 145 extending from body (or housing) 110.

Within body 110 are shown power supply 120 for providing a voltage to electronic circuitry 130. Power supply 120 may, for example, comprise one of: an alkaline battery or a rechargeable battery (Lithium Ion, Nickel Metal hydride, Nickel Cadmium, etc.). In another aspect of the invention, power source 120 may be an external power source (not shown), such as an AC/DC converter that may be connected to an electrical outlet. In this case, power supply 120 may further include step-down transformers and voltage converters that convert the known alternating current voltage available at the electrical outlet to a direct current electrical value that may be applied to electronic circuitry 130.

Electronic circuitry 130 may include resistors, capacitors, diodes, transistors, special application integrated circuitry (ASIC) and/or microcontrollers that are suitable for controlling the operation of Lighted Mirror System 100. Resistors, capacitors, diodes, transistor, etc. are well-known in the art and a detailed discussion of these elements is believed not necessary for those skilled in the art to practice the invention claimed.

Further illustrated is switch 140 that controls the application of a voltage from power source 120 to one or more of the illustrated lighting sources 155, 165. For example, in one aspect of the invention, switch 140 may comprise an on/off switch that causes voltage from power supply 120 to be applied (through electronic circuit 130) to lighting sources 155, 165 concurrently. In another aspect of the invention, switch 140 may comprise a multiple position switch that may provide voltage to lighting source 155, for example, when in a first position and voltage to lighting source 165 when switch 140 is in a second position and remove the voltage from lighting sources 155, 165 when in a third position. Alternatively, switch 140 may represent a switch through which power from power supply 120 is provided to electronic circuitry 130, wherein transistors (or switches) within electronic circuitry 130 may control the application voltage to one or both of lighting sources 155, 165. Control of the switches or transistors on electronic circuitry 130 may be performed by a remote switch, for example, that may be in wired, or in wireless, communication with electronic circuitry 130. For example, electronic circuitry 130 may include a near-field communication system (e.g., BLUETOOTH) that may be in wireless communication with a remote switch (not shown). Operation of the remote switch may be received through a near-field communication system to apply the voltage available on electronic circuitry 130 to one or both of lighting sources 155, 165.

In accordance with the principles of the dual-light lighted mirror system 100 disclosed, lighting sources 155 and 165 are positioned on a distal end of body 110 and arranged to emit light into a corresponding one of reflective mirror 150 and emission mirror 160.

Lighting sources 155, 165, as will be discussed, are comparable to lighting sources disclosed in one or more of U.S. Pat. Nos. 10,132,483; 10,215,977; 10,240,769; 10,247,384, 10,437,041; 10,527,254 and 10,895,735, the content of which are incorporated by reference, herein. More specifically, lighting sources 155, 165 may comprise a semiconductor light emitting die (or diode), an aperture holder, an aperture and a dome lens, to provide for the projection of more uniform and focused emitted light. Lighting source 155 and 165 are arranged to emit light in a corresponding known wavelength range.

Further illustrated is an emission mirror (e.g., a mirror, a polished metal surface, etc.) 160 positioned along arm 145 and a reflective mirror (e.g., mirror, polished metal surface) 150 positioned at a distal end of arm or rod 145.

In accordance with the principles of the invention, light emitted by lighting source 165 is directed toward emission mirror 160, which redirects the emitted light along light path 167 to an object (not shown). Similarly, light emitted by lighting source 155 is directed toward reflective mirror 150 which redirects the emitted light along light path 157 toward the not-shown object.

The light emitted by either lighting source 155 or lighting source 165 may illuminate the not-shown object, which may then reflect the illuminating light back toward reflective mirror 150/emission surface 160. The reflected light, representing an image of the light projected onto the not-shown object, may be captured by reflective mirror 150 along light path 170, which may then be viewable through reflective mirror 150.

In accordance with one aspect of the invention and for purposes of describing the invention, lighting source 155 may comprise a light source (e.g., a light emitting diode) suitable for emitting a white light (i.e., wavelengths in range of 380 nm to 700 nm). And lighting source 165 may comprise a light source suitable for emitting light in one or more of a plurality of wavelength ranges (i.e., Ultra-Violet (1 to 400 nm), visible (380 nm to 700 nm) and near infrared (700 nm to 1400 nm), individually or in combination.

Although reflective mirror 150 and emission mirror 160 are shown at substantially a same angle with respect to arm 145, it would be recognized that mirrors 150 and 160 may be oriented at different angles with respect to arm 145 such that light projected along path 167 converges at a point coincident with the light projected along path 157.

Although mirror 150 is referred to as “reflective mirror” and surface 160 is referred to as “emission mirror,” it would be recognized that mirror 150 and 160 may be both reflective and emissive and the terms “reflective” and “emissive” are used, herein, only to express the primary function of each surface. That is, it is intended that mirror 150, being the larger of the two surfaces, is used to view light reflected from the not-shown object even though light reflected from the object may be viewable in mirror 160. Similarly, mirror 150, in this exemplary embodiment, may be used as both as an emissive surface and a reflective surface.

In accordance with the principles of the invention, light emitted by, for example, lighting source 155, is directed toward mirror 150, which as discussed is a white light, allows for the illumination of an area or object to which the light from lighting source 155 is re-directed.

A practitioner then may view an image of the illuminated area in the reflective surface of mirror 150.

In addition, light emitted by lighting source 165, which may for purposes of describing the invention claimed, emit light in a different wavelength range. The light emitted by lighting source 165 is directed toward emissive mirror 160 and re-directed toward the area or object along path 167.

As is known in the art, components of certain pathogenic bacteria within mature biofilms have the capacity to emit light at certain wavelengths due to intrinsic components (e.g., porphyrins). Hence, light emitted in one or more known wavelengths emitted by light source 165 may cause bacteria (or infection) within the illuminated area to self-illuminate, wherein a light (a fluorescent light) in a wavelength different than that emitted by lighting source 165, may be generated. The intensity of the fluorescent light emitted by the illuminated infection or bacteria is determined based on the degree or level of infection.

Accordingly, a practitioner, through the appropriate selecting of lighting and filtering may distinguish fluorescent light (and the area generating such light) from the light emitted by lighting source 165.

Further illustrated is image capture device (e.g., a camera) 180 contained within blister assembly 182. Image capture device 180 captures and records images projected onto reflective mirror 150 for subsequent review and analysis of the area (not shown) being illuminated (and/or treated) by the lighted mirror system 100 shown.

Further illustrated within blister assembly 182 is filter 184. Filter 184 limits the light wavelengths captured by image capture device 180 to a desired viewable wavelength range.

For example, filter 184 may limit the wavelengths of light viewed by image capture device 180 to a wavelength range associated with the fluorescent light generated by the illumination of bacterial microbiome or bacterial biofilm exposed to emitted light of lighting source 165, for example.

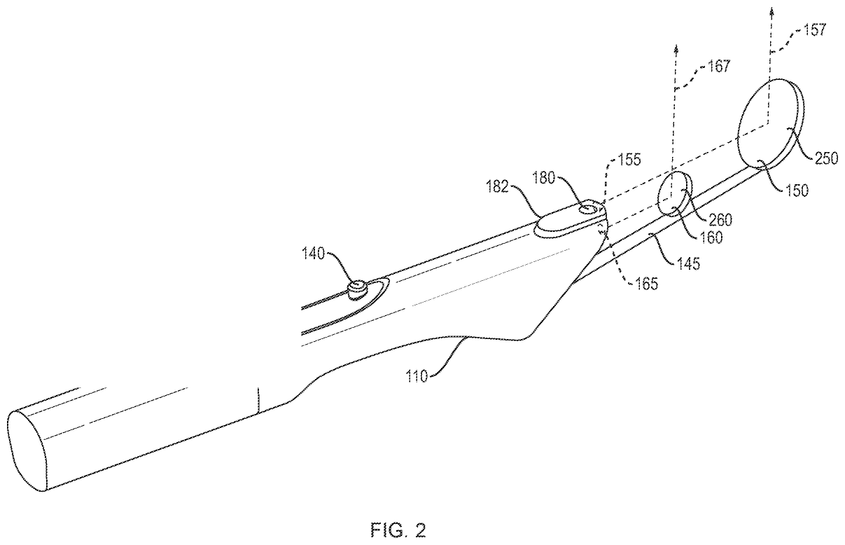

FIG. 2 illustrates a perspective view of the exemplary embodiment of lighted mirror system 100 shown in FIG. 1.

In this illustrated embodiment, light emitted by lighting source 155 is primarily directed toward mirror 150, which redirects the received light along light path 157. Similarly, light emitted by lighting source 165 is directed toward emission surface 160, which redirects the light along light path 167. Light emitted by light source 155 or 165 may be reflected by an object (not shown) and an image of the object may be viewed on surface 250 of reflective surface 150.

In one aspect of the invention, as light viewed on surface 250 of mirror 150 may include light emitted by one or both of lighting sources 155, 165 and the light emitted by one or both of lighting sources 155, 165 may be harmful if viewed by a practitioner, it may benefit the practitioner to provide a coating on surface 250 to provide for limiting the wavelength range of the light viewable on surface 250. That is, the coating applied to surface 250 may comprise optical (i.e., filtering) characteristics that attenuate light reflected from the object that is in a wavelength range harmful to a user's eyes, while allowing light not harmful to the user to be viewable.

Similarly, surface 260 of emission mirror 160 receiving light from lighting source 165 may include a coating or filter that provides for the limiting (or tailoring) of the wavelength range of the light reflected by surface 260 onto the object (not shown).

Further illustrated are image capture device 180 within blister assembly 182, wherein image capture device 180 may capture an image (or record) a video of an object illuminated by lighting sources 155, 165.

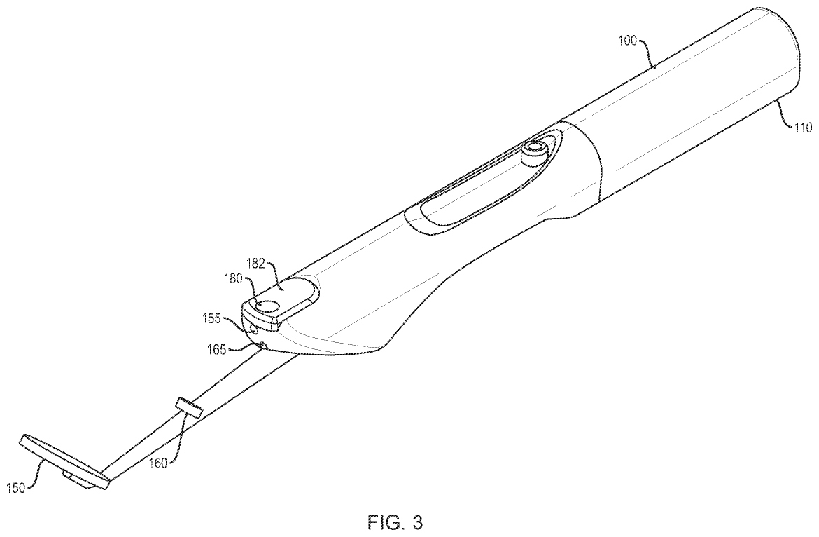

FIG. 3 illustrates a front perspective view of lighted mirror system 100 shown in FIG. 1.

In this exemplary embodiment, lighting sources 155 and 165 are incorporated into a distal end of body 110 such that the light emitted by lighting source 165 is directed toward emission mirror 160 and the light emitted by lighting source 155 is directed toward reflective mirror 150.

Further illustrated is image capture device 180 within blister assembly 182 for capturing images of the light reflected by the not shown object.

Although FIG. 3 illustrates lighting sources 155 and 165 positioned on a distal end of body 110, it would be recognized that lighting sources 155 and 165 may be internal to body 110 and the light emitted by lighting sources 155 and 165 may pass through windows or filters as the emitted light exits body 110. For example, assuming light source 155 emits a white light then the emitted light may pass through a clear window which allows all wavelengths to pass unattenuated and lighting source 165 emits a colored light (i.e., non-white or limited wavelength range), wherein the emitted light may pass through a transmissive filter that defines a range of wavelengths that is directed toward mirror 160. For example, assuming light from lighting source 165 is in a wavelength range of 450-475 nanometers (nm), then a transmissive filter may be selected to limit the wavelength range of light directed toward surface 160 to be in the wavelength range of 460-470 nm, for example.

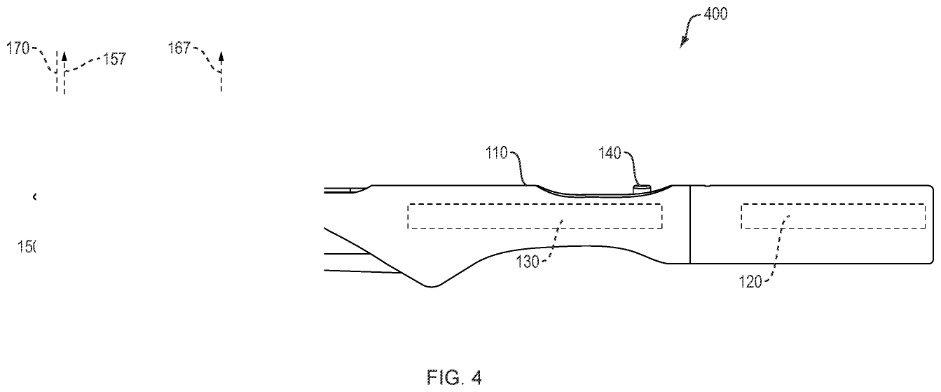

FIG. 4 illustrates a second exemplary embodiment of a lighted mirror system 400 in accordance with the principles of the invention.

In this second exemplary embodiment, lighted mirror system 400 similar to lighted mirror system shown in FIG. 1 comprises body 110, extension arm 145, reflective mirror 150, power supply 120 and electronic circuitry 130. Further illustrated are switch 140 and lighting source 155.

Lighting source 155, positioned on a distal end of body 110, projects light toward reflective mirror 150 (with or without a filter coating 250), which redirects the light along path 157, as previously discussed.

Further illustrated is second lighting source 165 positioned within a housing or blister 420 on extension arm 145, wherein light projected by second lighting source 165 is directed along light path 167.

Although light directed along light path 167 is shown substantially perpendicular to extension arm 145, it would be recognized that second lighting source 165 may, in one aspect of the invention, be oriented on at angle such that the light emitted by second lighting source 165 may converge with the light travelling along light path 157 at a known distance (e.g., 1.0-3.0 inches) from reflective mirror 150.

Housing or blister 420 may, for example, represent a clear window discussed previously. Alternatively, blister 420 may represent a transmissive filter that may limit the wavelength range of the light emitted by lighting source 165.

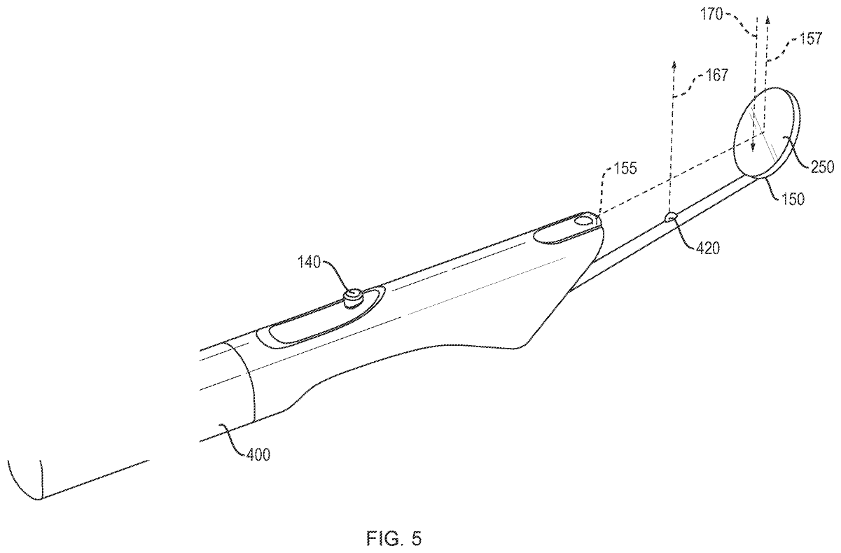

FIG. 5 illustrates a perspective rear view of the light mirror shown in FIG. 4

In this aspect of the invention, surface 250 of reflective mirror 150 may include a coating or filter that eliminates or reduces in magnitude light in at least one wavelength band from light travelling along light path 170 from being viewed by a user.

For example, light emitted by lighting source 165 within blister 420 may represent light in a wavelength range that is harmful if viewed by a practitioner. A coating on surface 250 may reduce the magnitude or intensity of light associated with lighting source 165 from being viewed when light associated by lighting source 165 is intercepted by surface 250.

In one aspect of the invention, the coating on surface 250 may comprise materials that reduce in magnitude light in one wavelength range (e.g., light harmful to a user) while allowing light in a second wavelength range to pass substantially unaltered.

Although not shown it would be recognized that image capture device 180 of FIG. 1 may be incorporated in the example embodiment of a lighted mirror system shown in FIGS. 4 and 5.

In an alternative embodiment, a transmissive filter may be applied to the light emitted by lighting source 155 to limit the wavelength range of the light emitted by lighting source 155 to a desired wavelength range.

In this alternative embodiment, lighting source 165 may be removed from the extension arm 145.

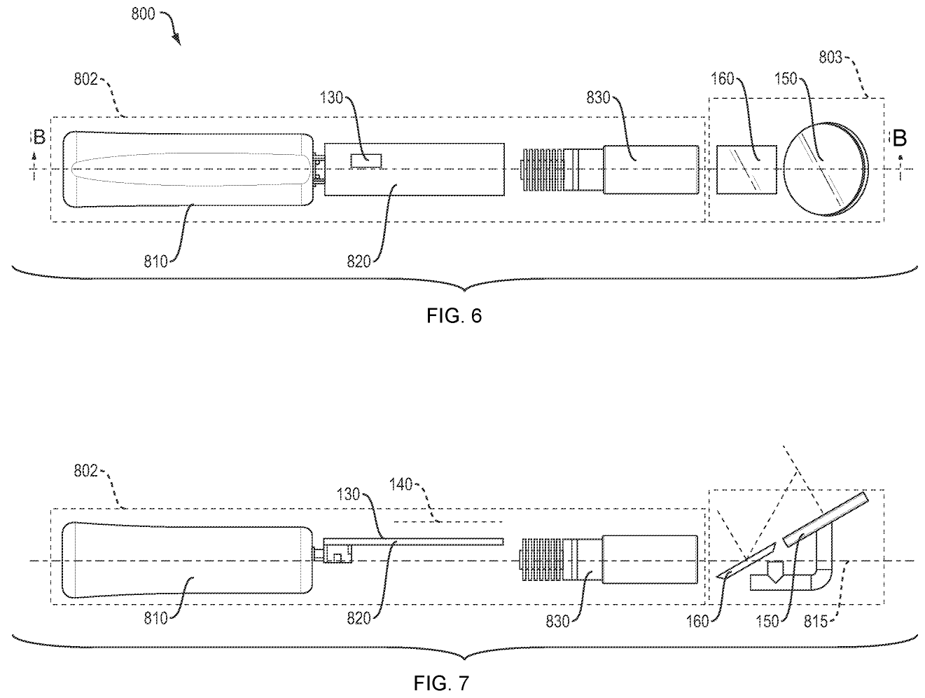

FIG. 6 illustrates a top view of a third exemplary embodiment of a lighted mirror system 800 in accordance with the principles of the invention, wherein a single lighting source (e.g., lighting source 165) is disclosed.

In this exemplary embodiment, lighted mirror system 800 comprises housing 802 and an insertable mirror section 803. Housing 802 and mirror section 803 are shown in dashed lines to show the internal elements, therein. Housing 802 comprises a body or handle section 810, a main control board 820, switch 140 and an optical assembly 830, which is shown separated from main control board 820. Mirror section 803, which may be removably attachable to housing 802 comprises an emissive mirror 160 and a reflective surface 150. Removable mirror section 803 is advantageous as it allows for the sterilization of mirror section 803 without requiring a full sterilization of lighted mirror system 800.

Main control board 820 comprises electronic circuitry 130 similar to that disclosed with regard to FIGS. 1 and 4 to control the light outputted by lighted mirror system 800.

FIG. 7 illustrates a side view, through section B-B of the top view shown in FIG. 6.

In this illustrated view, emission mirror 160 and reflective mirror 150 are arranged at an angle with respect to a horizontal axis 815 of lighted mirror system 800. In a preferred embodiment emission mirror 160 and reflective mirror 150 are oriented (arranged or angled) to cause light emitted by a light source within optical assembly 830 to converge at a known distance (not shown) substantially above reflective mirror 150.

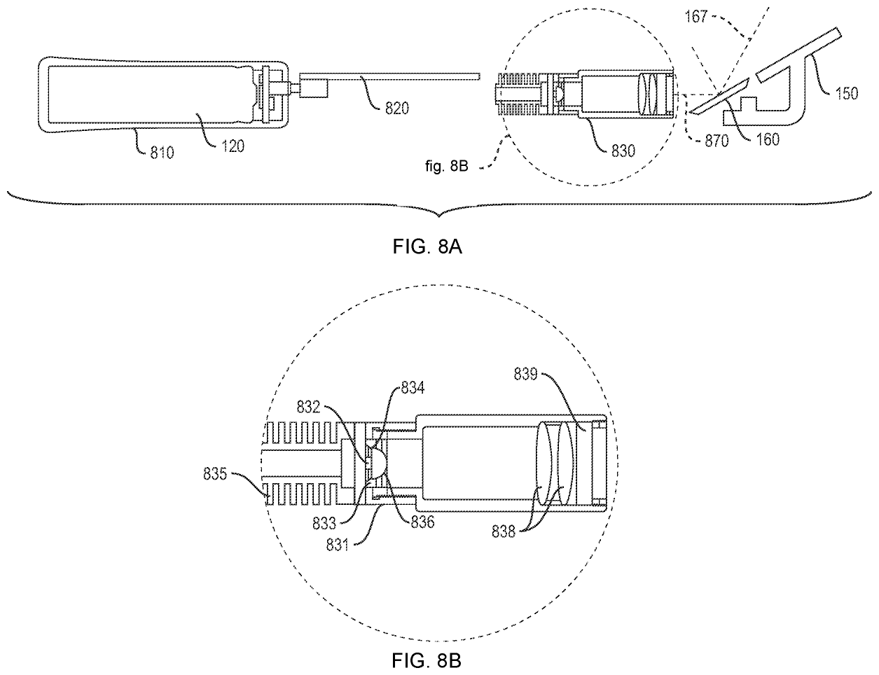

FIG. 8A illustrates a cut-away side view of the lighted mirror system shown in FIG. 7, wherein handle or body 810 is shown to include a power source (i.e., a battery) 120 that provides an electrical energy to electrical components (circuitry 130) contained main control board 820.

As discussed previously, electronic circuitry 130 provides voltage emitted by power supply 120 to, in this illustrative case, a lighting source (e.g., lighting source 165) within optical assembly 830, wherein emitted light is emitted along a substantially horizontal or longitudinal axis of body 802.

Further illustrated is the angle of first mirror 160, with respect to the emitted light is such that emitted light is directed at an angle, along light path 167, toward an object (not shown) a known distance (e.g., 1-2 inches) above reflective mirror 150.

FIG. 8B illustrates an enlarged cut-away side view of optical assembly 830, shown in FIG. 8A, comprising housing 831 including at least one objective lens 838 on a first (distal) end (closest to emission mirror 160) and heat sink 835 on a second end. Further illustrated is lighting source 165 comprising a light source 832 (i.e., at least one light emitting diode or semi-conductor die), an aperture holder 833, including an aperture holder passthrough (not shown), aperture 834, including an aperture passthrough (not shown) and a dome lens 836.

In one aspect of the invention, light source 832 may be positioned within a focal length of the dome lens 836 and within a focal length of objective lens 838. A more detailed discussion of the lighting source 165, referred to herein, may be found in U.S. Pat. Nos. 10,247,384 and 10,527,254, for example. Although the lighting configuration disclosed is the similar to the lighting configuration disclosed in U.S. Pat. Nos. 10,247,384 and 10,527,254, it would be understood, that the lighting configuration disclosed, herein, is only one example of a lighting configuration and other configurations are considered within the scope of the invention. For example, another exemplary lighting configuration that is considered within the scope of the invention may lack one or more of aperture holder 833, aperture 834 and dome lens 836. For example, when dome lens 836 is utilized, the light emitted by the lighting source may be more uniform and directed (i.e., a narrow beam of light). Whereas in another aspect of the invention, the lighting source may not include a dome lens and the light emitted by the lighting source may be more diverse (i.e., a wider beam of light). Similarly, the aperture holder 832 and aperture may be utilized to provide for a whiter light output.

Further illustrated is transmissive filter 839 positioned on a distal end of optical assembly 830. Transmissive filter 839 is configured to tailor the light outputted by lighting source 165, wherein tailoring in this case refers to adapting the light outputted by lighting source 165 to meet a desired wavelength range.

Objective lens 838 receiving light emitted by the light emitting diode 832, passes the received light to transmissive filter 839, which limits or filters the wavelength band of the emitted light. The filtered light is directed along optical path 870 toward emission mirror 160, wherein emission mirror 160 redirects the received light along light path 167 (see FIG. 8A), as previously discussed.

In one aspect of the invention, where the lighting configuration is such that a white light is emitted, transmission filter 839 may be configured to limit the outputted wavelength band (e.g., 390 nanometers (nm)-700 nm) to a desired wavelength band. For example, transmission filter 839 may represent a narrow bandpass filter having filtering characteristics that limit the light emitted by optical assembly 830 to a range between 400 and 430 nanometers. Alternatively, transmission filter 839 may be a low pass filter having filtering characteristics that limit the light emitted by optical assembly 830 to a range less than 430 nanometers. In this case, with an emitted white light (390-700 nanometers), transmission filter 839 limits the light emitted to be between 390-430 nanometers. Similarly, when the light source (LED) 832 is a source that emits light, for example, in a violet wavelength range (e.g., 400-450), transmission filter 839 may limit the wavelength range of emitted light to be within 400-430 (assuming the passband filter as previously discussed).

While a 400-430 nanometer filter is discussed, it would recognized that other filters and filter characteristics (wavelength bandpass ranges) may be utilized without altering the scope of the invention.

Further illustrated is heat sink 835, which directs heat generated by LED 832 away from LED 832.

In one aspect of the invention, optical assembly 830 and housing 810 may be removably attachable within housing 802, wherein optical assembly 830 and housing 810 may be attached through one of a screw thread connection, a bayonet connection, snap-fit connection and other similar type connectors.

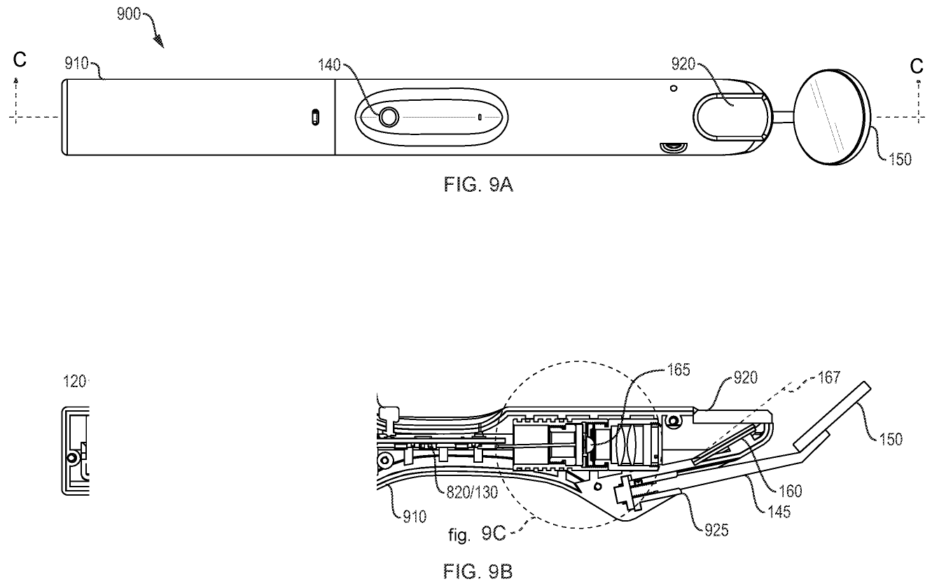

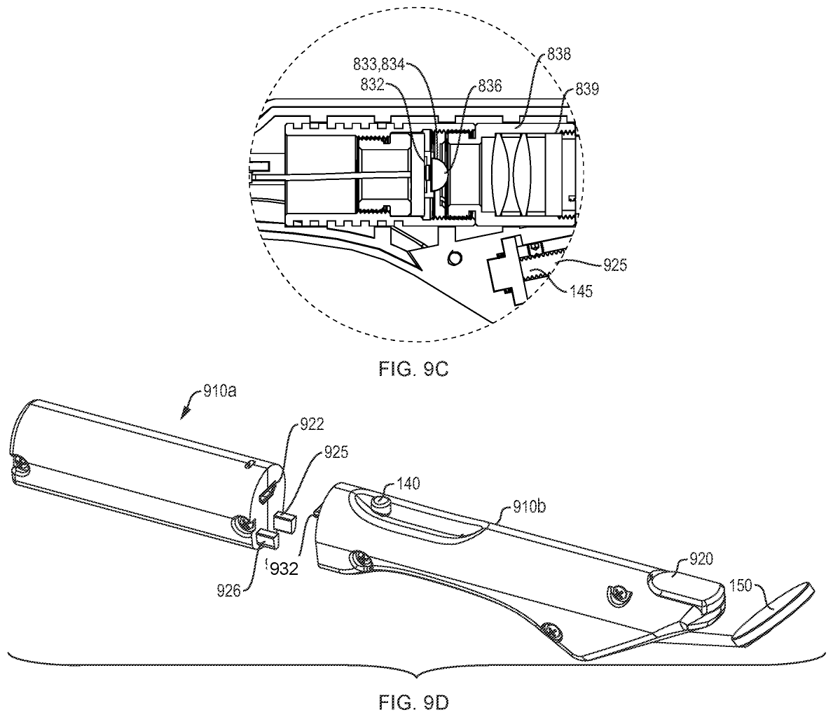

FIG. 9A illustrates a top view of a fourth exemplary embodiment of a lighted mirror system 900 in accordance with the principles of the invention.

Lighted mirror system 900 comprises housing 910 including switch 140 as previously discussed. Further illustrated is reflective mirror 150 extending from housing 910.

In accordance with this fourth exemplary embodiment, housing 910 includes window 920 positioned on a top surface of housing 910.

FIG. 9B illustrates a cutaway side view, through section C-C of the lighted mirror system 900 shown in FIG. 9A.

In this exemplary view, incorporated into housing 910 are power source 120 providing electrical energy, through switch 140 to PCB 820/electronic circuitry 130, as previously discussed. Electronic circuitry 130 controls the application of the voltage from power source 120 to electronic circuitry 130 and subsequently to lighting source 165. Further illustrated, within housing 910 is emission surface 160.

Further illustrated is reflective surface (e.g., second mirror) 150 extended from housing 910 by extension arm 145.

In one aspect of the invention, second mirror (reflective surface) 150 may be removably attachable to housing 910 at location 925 to allow for the sterilization of reflective surface (second mirror) 150.

In accordance with the principles of the invention, light generated by, in this illustrative example, lighting source 165 is directed toward reflective surface 160 and redirected along light path 167 in a manner similar to that discussed with regard to FIGS. 1, 4 and 8, for example. In this case, the re-directed light is directed toward an area substantially above second mirror 150, such that light reflected by an object (not shown) illuminated by the redirected light is viewed by reflective surface 150.

FIG. 9C illustrates an enlarged cut-away side of the lighting source shown in FIG. 9B, wherein the illustrated lighting source comprises a light source 832, aperture holder 833 aperture 834, and a dome lens 836.

Similar to the lighting source discussed with regard FIG. 8B, the lighting configuration disclosed in FIG. 9C is similar to the configurations disclosed in U.S. Pat. Nos. 10,247,384 and 10,527,254 as this provides a more uniform and directed emitted light. Similarly, it would be understood, that the lighting configuration disclosed with regard to FIG. 9C is only one example of a lighting configuration and other configurations are considered within the scope of the invention. For example, another exemplary lighting configuration may lack one or more of the aperture holder 832, aperture 833, dome lens 836, objective lens 838 and filter 839.

FIG. 9D illustrates a perspective view of lighted mirror system 900 in accordance with the principles of the invention.

in this illustrated embodiment, housing 910 comprises first housing 910a and second housing 910b, wherein first housing 910a comprises power source 120 (not shown) and second housing 910b comprises PCB 820/electronic circuitry 130, lighting source 165, for example, and window 920. Further illustrated is reflective mirror 150, which as discussed, is removably attachable to second housing 910b.

In one aspect of the invention, window 920 may be a clear window that allows light to pass-through unaltered. In another aspect of the invention window 920 may comprise a filter element (similar to filter 839) that limits the range of light emitted through window 920.

As shown, first housing 910b is removably attachable to second housing 910b, wherein first housing 910b includes a port 922 into which an electrical tab 932 on second housing 910b is insertable. In one aspect of the invention, port 922 may be a micro-USB port, for example, that allows the transfer of electrical energy from the power supply 120 (not shown) within housing 910a to PCB 820/electronic circuitry 130 (not shown) in second housing 910b.

Further illustrated are alignment/retention tabs 925, 926 on first housing 910a, which mate with matching ports (not shown) on second housing 910b and are configured to retain first housing 910a to second housing 910b with a snap-fit connection.

Although a snap-fit connection is shown, it would be known to those skilled in the art to incorporate other types of connections to retain first housing 910a to second housing 910b. For example, connection between first housing 910a and second housing 910b may comprise a bayonet connection, a screw thread connection, etc., wherein electrical energy from power supply 120 may be transferred between first housing 910a and 910b through a contact ring connection, for example.

While a micro-USB port is discussed, it would be well-known in the art to alter the use of a micro-USB port with other types of electrically connections. For example, a conventional USB connector, a power jack (i.e., power plug/power receptacle), barrel connector, USB Type connector, pin connector, a lightning connector (similar to that used on an Apple Corporation iPhone®), etc., have been contemplated and considered within the scope of the invention claimed. iPhone is a registered trademark of Apple inc. Corporation, California.

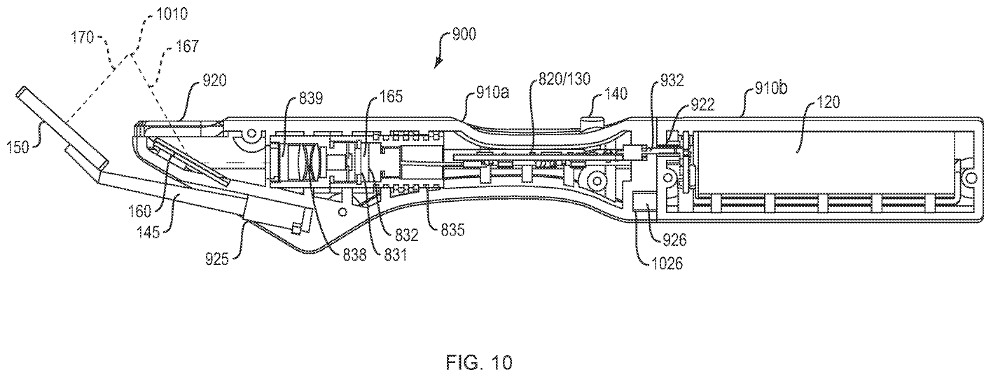

FIG. 10 illustrates a second side view of the fourth exemplary embodiment of the lighted mirror system 900 shown in FIG. 9A.

In this second side view of the fourth embodiment of the lighted mirror system 900 shown, similarly labeled elements would be understood from a reading of the description of those elements shown in FIG. 9A-9D.

In this illustrated view, first housing 910a is shown attached to second housing 910b, wherein electrical connection between PCB 820/electronic circuitry 130 and power supply or power source 120 is provided by the insertion of electrical tab 932 into electrical socket 920.

Further illustrated is alignment/retention tab 926 inserted into connection socket 1026.

Further illustrated are window 920, filter 839, optical housing 831 including lighting source 165, wherein lighting source 165 comprises, in this case, light source 832, and heat sink 835.

In this illustrated case, window 920 is substantially clear and filter 839 tailors the light emitted by lighting source 165 to a desired wavelength range. Further illustrated are reflective mirror 150 extending along shaft or extension arm 145, which is insertable into pocket 925 within second housing 910b.

In accordance another aspect of the invention, window 920 may include a coating that causes window 920 to operate as filter 839 to limit the wavelength range of the light emitted by lighting source 165. In this aspect of the invention filter 839 may not be necessary.

Further illustrated is emission mirror 160 receiving light from lighting source 165 (i.e., light source 832) through filter 839, which is emitted along substantially a horizontal (or longitudinal) axis of body 910 and redirecting the received light along light path 167 toward an object 1010, such as a tooth. As shown, emission mirror 160 is oriented to receive the substantially horizontally emitted light and direct the received light at an angle toward an object 1010. A determination of the orientation of emission mirror 160 would be understood by those skilled in the art using well-known Law of Reflection (i.e., light is reflected at a same angle, with respect to a normal to the reflective surface, as the incident angle, with respect to the normal to the reflective surface).

Light reflected from object 1010 may be received by reflective mirror 150 (along light path 170). The reflected light may subsequently be viewable by a practitioner.

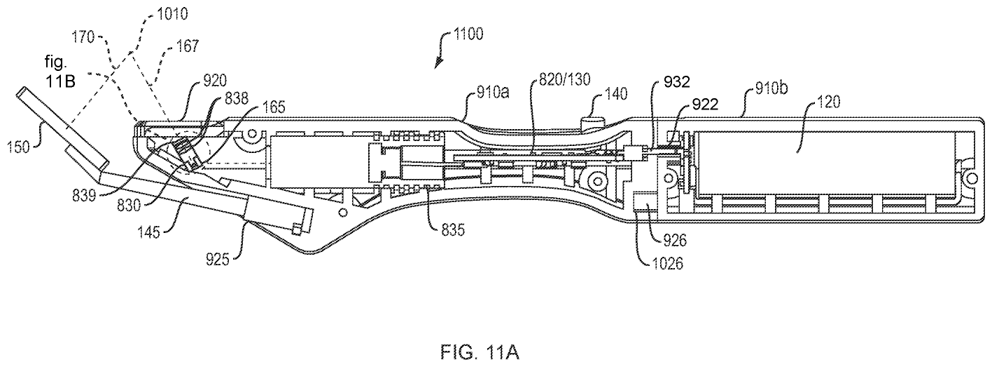

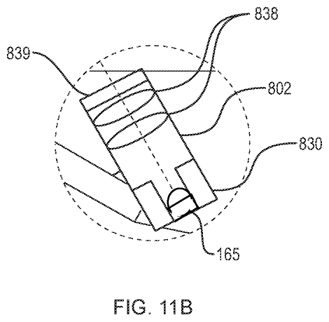

FIG. 11A illustrates a side view of a second aspect of the fourth embodiment of lighted mirror system shown in FIG. 9A in accordance with the principles of the invention.

In this second aspect of the fourth embodiment of the lighted mirror system (referred to as 1100) similarly labeled elements are comparable to those element shown and disclosed with regard to FIGS. 9A-9D and 10 and the operation of this second aspect of the fourth embodiment of the lighted mirror system shown in FIG. 11 would be understood from a reading of the description of those elements shown in FIGS. 9A-9D and 10

In this aspect of the invention, reflective mirror 160 is removed and replaced with optical assembly 830, which is oriented at an angle to emit light along light path 167.

In this illustrated second aspect, optical assembly 830 comprises at least one of: at least one objective lens 838 and lighting source 165, wherein lighting source 165 comprises at least light source 832, wherein elements aperture holders 833, aperture 834 and dome lens 836 may be optionally included.

Further shown is window 920 which in this aspect comprises optical material that causes window 920 to operate as a filter to limit the light outputted by lighting source 165 to a desired wavelength range.

FIG. 11B is an expanded view of the area designated FIG. 11B shown in FIG. 11A, wherein optical assembly 830 is positioned within housing 910a at an angle to light emitted by lighting source 165 to be emitted along light path 167.

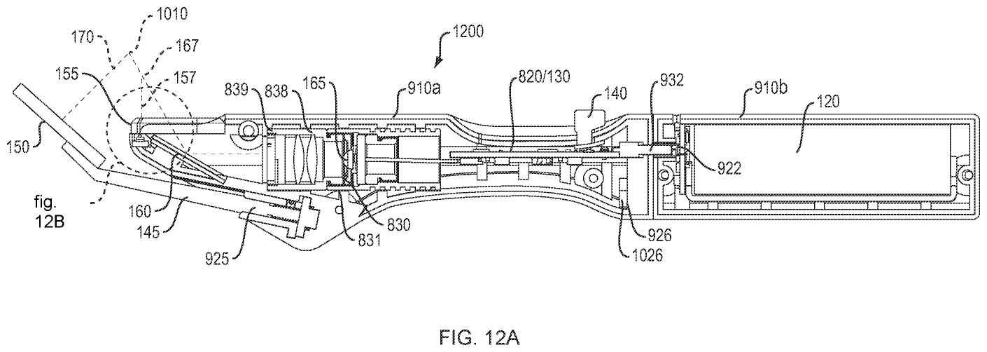

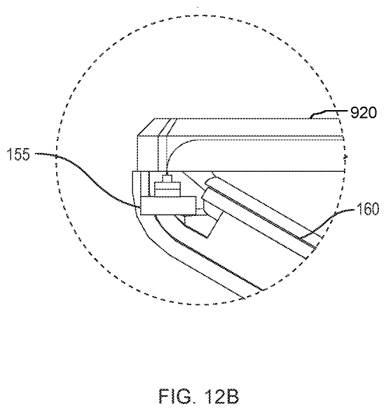

FIG. 12A illustrates a side view of a third aspect of the fourth embodiment of a lighted mirror system shown in FIG. 9A in accordance with the principles of the invention.

In this third aspect of the fourth embodiment of the lighted mirror system (referred to as 1200) similarly labeled elements are comparable to those element shown and disclosed with regard to FIGS. 9A-9D and 10 and the operation of this third aspect of the fourth embodiment of the lighted mirror system shown in FIG. 12A would be understood from a reading of the description of those elements shown in FIGS. 1, 9A-9D and 10

In this aspect of the invention, lighting source 155 is shown positioned at a distal end of second housing 910b, wherein light emitted by lighting source 155 is directed along light path 157 upward in a general vicinity of object 1010. Lighting source 155, in a preferred embodiment of this aspect of the invention, comprises a configuration wherein the previously discussed dome lens, similar to dome lens 836 is not included and light emitted by lighting source 155 is more diverse.

Further illustrated is lighting assembly 831 comprising lighting source 165, which in a preferred embodiment of this aspect of the invention comprises at least light source 832 and dome lens 836, to emit a focused light toward emission mirror 160, which redirects the emitted light toward object 1010 along light path 167, as previously disclosed.

As discussed, light emitted by lighting source 165 reflected by object 1010 may be viewed through reflection surface (i.e., mirror) 150.

In one aspect of the configuration shown in FIG. 12A, the light emitted by light sources 155 and 165 may be emitted concurrently or individually.

FIG. 12B illustrates an expanded view of the area designated FIG. 12B shown in FIG. 12A, wherein light source 155 emits light along light path 157 and light emitted by light source 165 (not shown) is directed by surface 160 along light path 167.

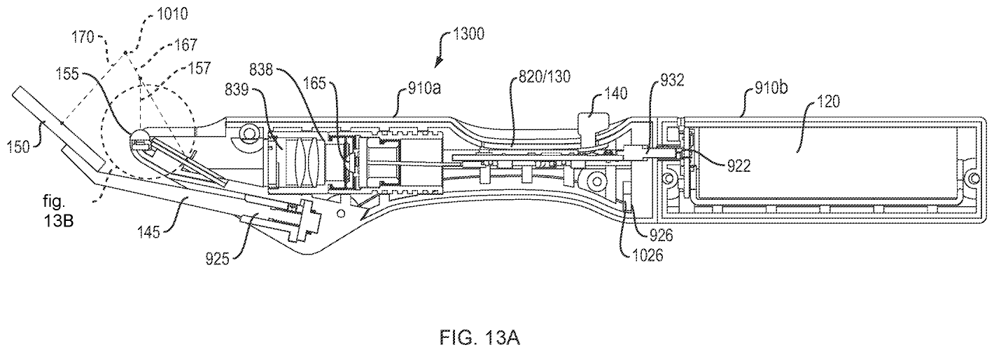

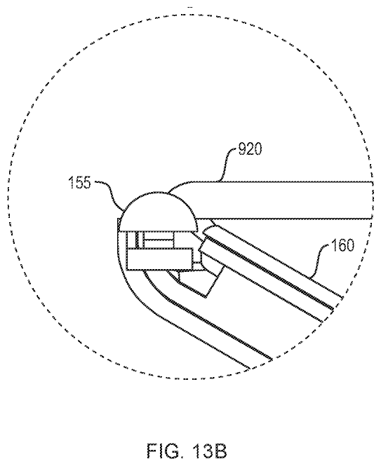

FIG. 13A illustrates a side view of a fourth aspect of the fourth embodiment of a lighted mirror system shown in FIG. 9A in accordance with the principles of the invention.

In this fourth aspect of the fourth embodiment of the lighted mirror system (referred to as 1300) similarly labeled elements are comparable to those element shown and disclosed with regard to FIGS. 9A-9D and 10 and the operation of this fourth aspect of the fourth embodiment of the lighted mirror system shown in FIG. 13A would be understood from a reading of the description of those elements shown in FIGS. 1, 9A-9D and 10.

In this illustrated aspect, lighting source 155 comprises a configuration as disclosed in U.S. Pat. No. 10,247,384, for example, is positioned in front of window 920, wherein light emitted by lighting source 155 is directed upward toward a general vicinity of object 1010. In this case, the light emitted by lighting source is focused and uniform.

In addition, light emitted by lighting source 165, which is similar to that shown in FIG. 11A, emits a focused and uninform light toward emission surface 160, which redirects the light toward object 1010.

As discussed previously, light emitted by lighting source 165 may be reflected by object 1010 and viewed through surface 150 (not shown).

FIG. 13B illustrates an expanded view of the area designated FIG. 13B shown in FIG. 13A, showing the positioning of lighting source 155 projecting a focused and more uniform light along light path 157.

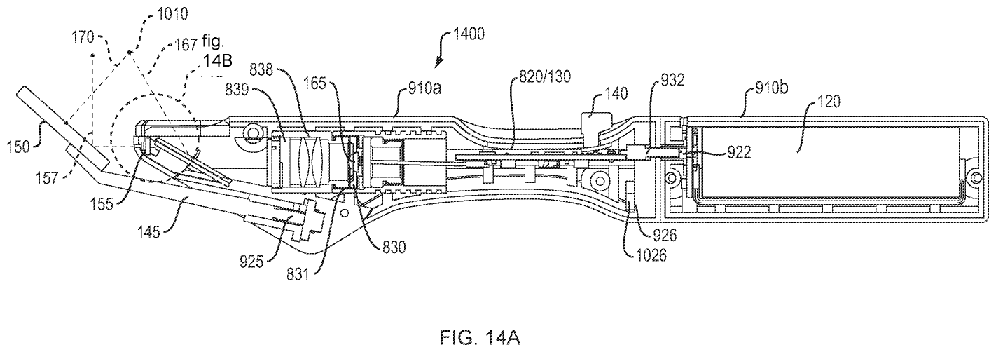

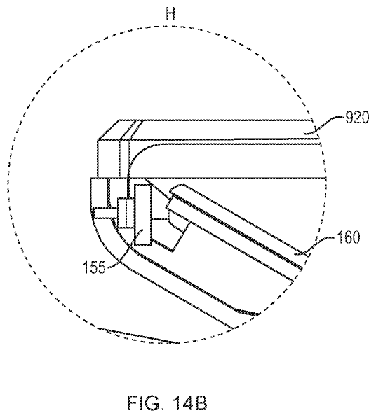

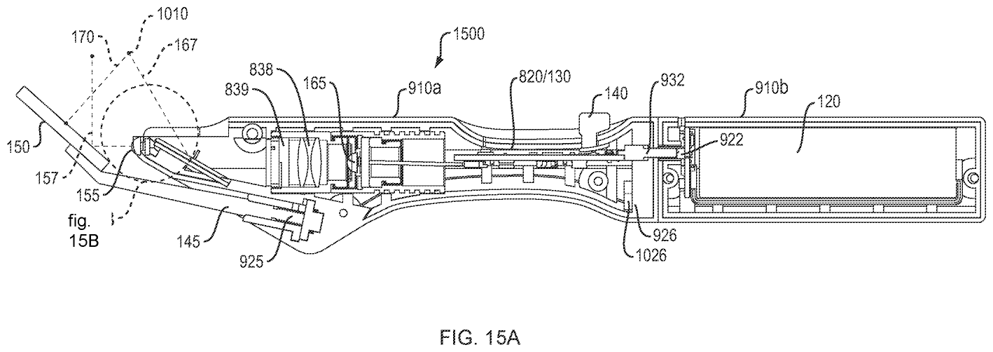

FIG. 14A illustrates a side view of a fifth aspect of the fourth embodiment of a Lighted Mirror System shown in FIG. 9A in accordance with the principles of the invention.

In this fifth aspect of the fourth embodiment of the lighted mirror system (referred to as 1400) similarly labeled elements are comparable to those element shown and disclosed with regard to FIGS. 1, 9A-9D and 10 and the operation of this fifth aspect of the fourth embodiment of the lighted mirror system shown in FIG. 14A would be understood from a reading of the description of those elements shown in FIGS. 1, 9A-9D and 10.

In this illustrated aspect, which is similar to that shown in FIG. 12A, lighting source 155 is positioned at a distal end of housing 910a in front of window 920, wherein light emitted by lighting source 155 is directed toward reflective mirror 150 and redirected in a general area of object 1010. On this illustrated example, lighting source 155 lacks a dome lens that would focus the emitted light.

Further illustrated is lighting source 165 emitting light toward emission surface (mirror) 160, which directs the emitted light toward object 1010 along light path 167.

As discussed previously, light emitted by first lighting source 165 is reflected by object 1010 and viewed through reflective surface 150.

FIG. 14B illustrates an expanded view of the area designated FIG. 14B shown in FIG. 14A, showing the positioning of lighting source 155 projecting light toward surface 150, which redirects the projected light along light path 157 (not shown). In this illustrated expanded view, lighting source 155 lacks a lens that would focus the emitted light.

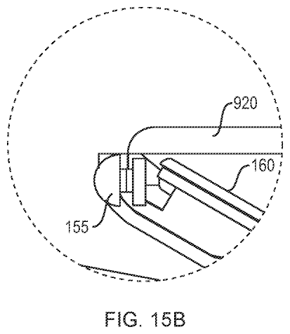

FIG. 15A illustrates a side view of a sixth aspect of the fourth embodiment of a lighted mirror system shown in FIG. 9A in accordance with the principles of the invention.

In this sixth aspect of the fourth embodiment of the lighted mirror system (referred to as 1500) similarly labeled elements are comparable to those elements shown and disclosed with regard to FIGS. 1, 9A-9D and 10 and the operation of this sixth aspect of the fourth embodiment of the lighted mirror system shown in FIG. 15A would be understood from a reading of the description of those elements shown in FIGS. 1, 9A-9D and 10.

In this aspect of the invention, lighting source 155, which is similar in construction to the lighting source 155 shown in FIG. 13A, includes a dome lens to focus the light emitted by lighting source 155 onto reflective mirror 150. Reflective surface 150 then directs the light emitted by lighting source 155 in the general area of object 1010.

Lighting source 165, as previously discussed, emits a focused light toward emission mirror 160, which directs the emitted light toward object 1010 along light path 167.

As discussed previously, light emitted by lighting source 165 is reflected by object 1010 and viewed through reflective surface 150.

FIG. 15B illustrates an expanded view of the area designated FIG. 15B shown in FIG. 15A, showing the positioning of lighting source 155 on a distal end of housing 910B projecting a focused and more uniform toward surface 150 (not shown). In this case, lighting source 155 includes a lens that is suitable for focusing the emitted light.

Although the lighted mirror system disclosed, herein, refers to lighting source 155 emitting a white light and lighting source 165 emitting a light in a wavelength range (e.g., ultra-violet, blue, green, etc.) it would be recognized that lighting source 155 may emit light in a wavelength range that is associated with a specific viewable color. For example, a blue wavelength range. Similarly, lighting source may emit light in a same wavelength range as that of lighting source 165.

Furthermore, although configurations of the lighted mirror system have been disclosed with regard to a single lighting source and two lighting source, it would be recognized that the number of lighting sources may be increased without altering the scope of the invention.

Although not shown it would be recognized that image capture device 180 of FIG. 1 may be incorporated in the example embodiment of a lighted mirror system shown in FIGS. 6-15, without altering the scope of the invention claimed.

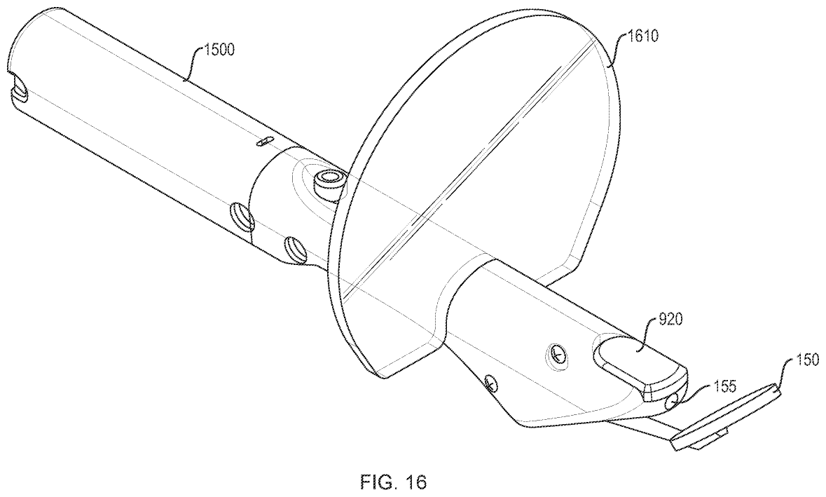

FIG. 16 illustrates a perspective view of a filter assembly configured to attach to Lighted Mirror System 1500, for example, shown, herein.

In this illustrated aspect, light emitted by at least one of lighting source 155, 165, for example, that may be harmful to the eyes of a user and, hence, requires the light emitted by lighting source 155/165 from being viewed by a user through mirror 150.

In this exemplary embodiment, reflective filter 1610 is fitted, using for example, a snap-fit connection, onto the illustrated lighted mirror system. In this illustrated example, lighted mirror system 1500 is depicted. However, it would be recognized that said reflective filter 1610 may be fitted to any of the different embodiments and aspects of the lighted mirror system disclosed, herein.

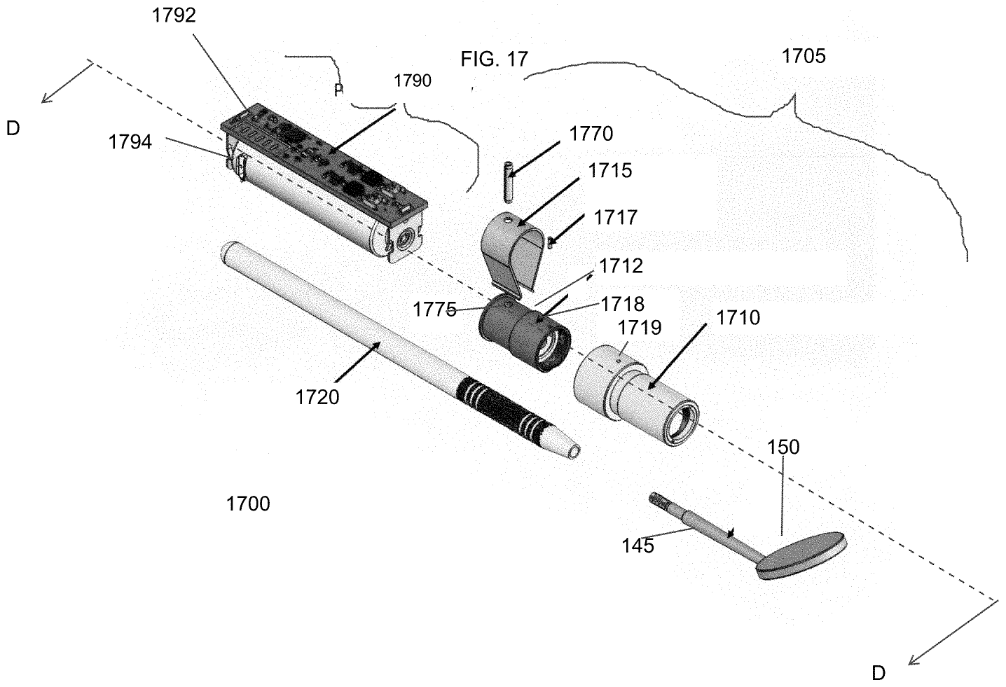

FIG. 17 illustrates of an exploded perspective view of a sixth exemplary embodiment of a Lighted Mirror system in accordance with the principles of the invention.

Lighted Mirror system 1700 comprises a handle 1720 into which may be inserted, through the extension arm 145, a reflective surface (mirror) assembly 150. Extension arm and mirror 150 are being removable attachable to handle 120 to allow for the sterilization of assembly 150.

Further illustrated is lighting assembly 1705 and battery assembly 1790, which are removably attachable to handle 120. Battery assembly 1790, in this illustrated example, comprises a printed circuit board (PCB) 1792 onto which are placed electronic components that control the application, or supplying, an electrical energy (i.e., voltage/current) to a lighting assembly 1705. The electronic components positioned on PCB 1792 comprise well-known components such as resistors, capacitors, diodes, transistors integrated circuits (i.e., Application Specific Integrated Circuits or Field Programmable Gate Arrays), which may form one or more control circuits that control the application of a voltage to lighting assembly 1705. Further illustrated is battery 1794 (e.g., conventional alkaline, rechargeable) that represents the source of electrical energy that is to be provided to lighting assembly 1705.

Although not shown, it would be recognized that a stand-alone, remotely located, source of electrical energy may be used to provide electrical energy to lighting assembly 1705. Such stand-alone, remotely located source of electrical energy may provide voltage/current to lighting assembly 1705 through a wired connected. Such stand-alone, remotely located source of electrical energy are well known in the art and would be an obvious modification to the embodiment of the invention disclosed, herein.

Lighting assembly 1705 comprises lens assembly 1710 and light housing 1712, wherein light housing 1712 may by inserted into a proximal end of lens assembly 1710. Lens assembly 1710 and light housing 1712 may be retained in place by the insertion of locking pin 1717 into passthroughs 1719 and 1718 on lens assembly 1710 and light housing 1712, respectively.

Further illustrated is spring clip 1715, which engages light housing 1712 and connects to handle 1720. Spring clip 1715 provides for the retention of lighting assembly 1705 onto handle 1720.

Although a spring clip 1710 is shown, it would be recognized that other forms of retention may be incorporated without altering the scope of the invention claimed. For example, retention means 1715 may comprise a screw thread configuration, wherein lighting assembly 1705 includes a screw thread that may be attached to a threaded post (not shown) extending from handle 1720. Similarly, retention means 1715 may include a locking clip that extends from one end lighting assembly 1705 under handle 1720 and attaches to a second end of lighting assembly 1705. In still another embodiment lighting assembly 1705 may be retained onto handle 1720 by replacing locating pin 1770 by a screw connection that engages a threaded passthrough within handle 1720. In still another embodiment, retention means 1715 may comprise a bayonet connection that attaches to locking pegs within handle 1720.

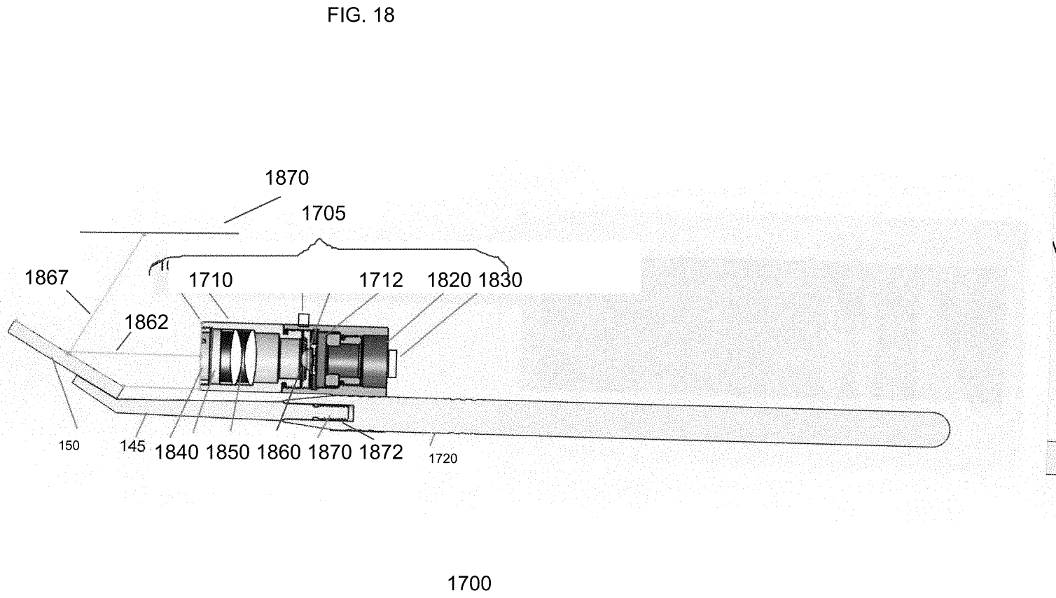

FIG. 18 illustrates a cross-sectional view, through section D-D, of the sixth exemplary embodiment of the Lighted Mirror system shown in FIG. 17 in accordance with the principles of the invention.

In this illustrated embodiment, light housing 1712, comprises a heat transferable material (e.g., stainless steel, aluminum, etc.) in contact with handle 1720, wherein light housing 1712 operates as a heatsink to transfer heat from a light generating element to handle 1720. Further illustrated is electrical contact 1830 through which electrical energy from a not-shown battery element (e.g., battery 195, FIG. 1) is provided to a light emitting assembly 1860. Further illustrated is lens assembly 1710 including light emitting assembly 1860, lens assembly 1850 and closing ring 1840 that closes off the distal end of lighting assembly 1705. Closing ring 1840 may comprise a plano lens that prevents dust or dirt from entering lighting assembly 1705 and prevents damage to the lens within lens assembly 1850.

Further illustrated is extension rod 145 attached to mirror 150 wherein extension rod (arm 144) comprises treaded end 1870 that may be screwed into a threaded pocket 1872 within handle 120.

In accordance with the principles of the invention, light emitted by lighting assembly 1860 is directed toward reflective surface 150 along optical path 1862, which is re-directed along optical path 267 toward a plane (or object) 270.

Lighting assembly 1860, similar to lighting assembly 830 comprises a lighting source that emits light substantially parallel to handle 1720 toward mirror 150. Lighting assembly 1860, similar to lighting assembly 830 comprises a lighting source, and/or one of more of an aperture holder, aperture and transmission filter as previously disclosed. Details of the operation and construction of lighting assembly 1860 would be understood from the discussing regarding lighting assembly 830.

For example, light assembly 1860 may be configured to emit light in at least one of a plurality of light wavelength ranges. For example, light emitted by light emitting assembly 1860 may emit light in one of an ultra violet wavelength range, a visible light wavelength range or an infra-red wavelength range. For example, light emitted in a visible light wavelength range may be limited to a specific wavelength range (e.g., blue, green, yellow, orange, red) or a white wavelength range (e.g., blue to red wavelength range) through the use of filters, as previously discussed.

In accordance with the principles of the invention, lighting sources may be incorporated into an optical assembly that provides for a narrow width (i.e., focused) light in a first wavelength range, wherein the first wavelength range may be in an ultra-violet wavelength range (e.g., 10-410 nm) and/or a blue wavelength range (e.g., 380-460) that cause the generation a fluorescent light by bacteria or infection when illuminated by the emitted light. The fluorescent light may then be viewed in a mirror assembly that may be inserted into a cavity (e.g., oral cavity).

The use of fluorescent light technology provides the practitioner with a means of viewing bacteria and/or infection that may not be visible.

In still another aspect of the invention, the exemplary embodiments and aspects of the lighted mirror systems disclosed, herein, FIG. 1 illustrates a camera or other similar type of image capture system that allows for the capturing of images (or videos) of the area illuminated by one or more of lighting sources 155/165. Captured images may then be transmitted to an external storage medium or stored in an internal storage medium. Although only the configuration shown in FIG. 1 illustrates an image capture system, it would be recognized that the other configuration of the lighted mirror system shown, herein, may include an image capture or camera system.

In summary, embodiments of lighted mirror systems are disclosed that comprises a handle and at least one lighting source, wherein light emitted by the at least one lighting source is directed toward an object and light reflected by the object, which may include a fluorescent light, may be viewed through a reflective surface that extends from the handle. In one aspect of the invention, the at least one lighting source positioned within the house to emit light directly toward the object. In another aspect of the invention, at least one lighting source is positioned to direct light an emissive surface that redirects light toward the object.

In one aspect of the invention, the lighted mirror system comprises an emissive mirror that operates as a transmissive surface to redirect the emitted light toward the object and a reflective mirror that operates as a reception surface that collects light reflected by the object and presents the collected reflective light to the practitioner.

In one aspect of the invention, the reception surface may include a coating that operates as a filter to prevent wavelengths emitted by the lighting source from being viewed.

In one aspect of the invention, the lighted mirror system may comprise a second lighting source that emits light in a second wavelength band, where the light emitted by the second lighting source may be directed to toward either the emissive mirror or the reflective mirror.

In one aspect of the invention, the lighting source may comprise a light source, such as a light emitting diode die to emit light in a known wavelength band.

In one aspect of the invention, a filter may be included that tailors the wavelength of the light emitted by the lighting source.

In one aspect of the invention, the lighting source may comprise a light source and a dome lens that focuses the light emitted by the light source.

The lighted mirror system presented, herein, allows for the use of FET technology to be utilized by practitioners for the examination of a patient's body cavities and the diagnosis of conditions within the body cavity. In addition, the mirror system disclosed may provide for the determining the effectiveness of treatment by allowing for the comparison of images of the body cavity at various times.

For example, in the dental arts, a practitioner may perform a tooth extract based one or more conditions either disclosed by the patient or uncovered by the practitioner. Images of the area of the extracted tooth may then be captured at various times to enable the practitioner to determine whether there is any residual or undisclosed infection and/or the rate of progress of removing any infection and/or healing of the area.

Although the example discussed above is related to the dental arts, it would be understood and recognized that the invention disclosed, herein, is not limited to the dental arts, but may be utilized in other fields without altering the scope of the invention claimed.

As used herein, the terms “comprises”, “comprising”, “includes”, “including”, “has”, “having”, or any other variation thereof, are intended to cover non-exclusive inclusions. For example, a process, method, article or apparatus that comprises a list of elements is not necessarily limited to only those elements but may include other elements not expressly listed or inherent to such process, method, article, or apparatus. In addition, unless expressly stated to the contrary, the term “of” refers to an inclusive “or” and not to an exclusive “or”. For example, a condition A or B is satisfied by any one of the following: A is true (or present) and B is false (or not present); A is false (or not present) and B is true (or present); and both A and B are true (or present).

The terms “a” or “an” as used herein are to describe elements and components of the invention. This is done for convenience to the reader and to provide a general sense of the invention. The use of these terms in the description, herein, should be read and understood to include one or at least one. In addition, the singular also includes the plural unless indicated to the contrary. For example, reference to a composition containing “a compound” includes one or more compounds. As used in this specification and the appended claims, the term “or” is generally employed in its sense including “and/or” unless the content clearly dictates otherwise.

All numeric values are herein assumed to be modified by the term “about,” whether or not explicitly indicated. The term “about” generally refers to a range of numbers that one of skill in the art would consider equivalent to the recited value (i.e., having the same function or result). In any instances, the terms “about” may include numbers that are rounded (or lowered) to the nearest significant figure.

Although the invention disclosed herein discusses specific wavelengths that are produced with currently available LEDs (i.e., non-lasing light emitting diodes and laser diodes), it would be recognized that the specific wavelengths absorbed and/or reflected may be changed and/or added to without altering the scope of the invention. In addition, it would be known in the art that the specific wavelengths discussed, herein, represent a band of wavelengths centered on the wavelength values presented herein to account for divergence of the wavelength generated by the light source during the generation of the light and/or the operation of the light source, wherein the light generated is represented as a nominal value.

The invention has been described with reference to specific embodiments. One of ordinary skill in the art, however, appreciates that various modifications and changes can be made without departing from the scope of the invention as set forth in the claims. Accordingly, the specification is to be regarded in an illustrative manner, rather than with a restrictive view, and all such modifications are intended to be included within the scope of the invention.

Benefits, other advantages, and solutions to problems have been described above with regard to specific embodiments. The benefits, advantages, and solutions to problems, and any element(s) that may cause any benefits, advantages, or solutions to occur or become more pronounced, are not to be construed as a critical, required, or an essential feature or element of any or all of the claims

What is claimed is:

1. A Lighted Mirror System comprising:
a body comprising:
a window;
an optical assembly comprising:
an optical housing, said optical housing containing, therein:
at least one objective lens; and
a lighting source, said lighting source configured:
to emit a light in a desired wavelength range toward said at least one objective lens, wherein said light is emitted through said window toward an object, and
a reflective surface extending from said body on an arm, said reflective surface configured to receive light reflected by said object.

2. The Lighted Mirror System of claim 1, said lighting source comprising;
at least one of: an aperture holder, an aperture and a dome lens.

3. The Lighted Mirror System of clam 1, wherein said at least one light source is configured to emit said light in one of: an ultra-violet (UV) light wavelength range, at least one visible light wavelength range, and an infra-red (IR) wavelength range.

4. The Lighted Mirror System of claim 1, comprising:
an emissive surface configured to:
receive said emitted light; and
direct said received emitted light toward said object.

5. The Lighted Mirror System of claim 4, wherein said emissive surface is:
internal to said body, wherein said window is positioned on a top surface of said body.

6. The Lighted Mirror System of claim 4, wherein said emissive surface is positioned along said arm external to said body.

7. The Lighted Mirror System of claim 2, comprising:
a transmissive filter configured to:
adapt a wavelength range of said emitted light to a desired wavelength range.

8. The Lighted Mirror System of claim 1, wherein said body comprises:
a first section comprising:
a power supply; and
a second section comprising:
said optical assembly, wherein said first section is removably attachable to said second section.

9. The Lighted Mirror System of claim 1, wherein said reflective surface is removably attachable to said body.

10. The Lighted Mirror System of claim 1, wherein said window is one of:
transparent and partially opaque, wherein said partially opaque window is limits said emitted light to a desired wavelength range.

11. The Lighted Mirror System of claim 1, comprising:
a reflective filter comprising characteristics configured to:
block from said light reflected by said object, said light emitted by said lighting source.

12. The Lighted Mirror System of claim 11, wherein said reflective filter is attachable to said body.

13. The Lighted Mirror System of claim 11, wherein said reflective filter characteristics are applied to said reflective surface.

14. A Lighted Mirror system comprising:
a body comprising:
a first body section comprising:
a power source
a second body section, removable attachable to said first body section, said second body section comprising:
a first lighting source configured to:
emit a first light in a first wavelength range;
a second lighting source configured to:
emit a second light in a second wavelength range, said first light and said second light being directed toward an object; and
an electrical circuity configured to:
apply a voltage from said power source to at least one of said first lighting source and said second lighting source; and
a reflective mirror extending from said second body section, said reflective mirror configured to:
receive a reflection of at least one of said first light and said second light from said object, said reflection comprising a fluorescent light generated in response to said object being illuminated by said first light.

15. The Lighted Mirror System of claim 14, comprising:
an emissive surface configure to:
receive said first light; and
direct said first light toward said object.

16. The Lighted Mirror System of claim 14, wherein said emissive surface is one of:
internal to said second body section and external to said body.

17. The Lighted Mirror System of claim 14 comprising:
a filter configured to:
block said first light in said reflected light from being view, and
allow said fluorescent light in said reflected light to be viewed.

18. The Lighted Mirror System of claim 17, wherein said filter is attachable to said body.

19. The Lighted Mirror System of claim 17, wherein said filter is incorporated onto said reflective surface.

20. The Lighted Mirror system of claim 1, comprising:
an image capture device configured to:
capture images reflected from said reflective surface.
